(19) United States
(12) Patent Application Publication
(10) Pub. No.: US 2014/0235730 A1
(43) Pub. Date: Aug. 21, 2014

(54) SOLID, EDIBLE, CHEWABLE LAXATIVE COMPOSITION

(71) Applicant: Gavis Pharmaceuticals, LLC, Somerset, NJ (US)

(72) Inventors: Veerappan Sellappan Subramanian, Mendham, NJ (US); Paranjothy Kanni, Mendham, NJ (US); Shyam Sundar Kumar Sarma Vangala, Pine Brook, NJ (US)

(21) Appl. No.: 14/346,668

(22) PCT Filed: Sep. 21, 2012
(86) PCT No.: PCT/US2012/056650

§ 371 Date: Mar. 21, 2014

Related U.S. Application Data
(60) Provisional application No. 61/538,488, filed on Sep. 23, 2011.
(60) Provisional application No. 61/620,688, filed on Apr. 5, 2012.

(51) Int. Cl. A23G 3/36; A23G 1/32; A23L 1/30; A61K 9/00; A61K 31/08

(52) U.S. Cl. CPC A23G 3/364; A61K 9/0056; A61K 31/08; A23L 1/30; A23G 1/32
USPC 514/723

(57) ABSTRACT

The invention is directed to an edible solid composition having laxative properties. The composition has a smooth mouth feel, in non-gritty and has a pleasant taste. It is prepared by melt-combining together the ingredients including a bulking agent and laxative as well as an optional flavor agent and electrolyte. The melt is a dispersion containing a substantially homogeneous combination of ingredients. The melt is cooled to form an amorphous solid dispersion or solution of the ingredients. The ingredients are mutually compatible so that upon cooling they do not separately crystallize to form a gritty mouth feel product.

### CLAIM OF PRIORITY

This application claims the benefit of priority of U.S. Provisional Application No. 61/538,488, filed Sep. 23, 2011, entitled “SOLID, EDIBLE, CHEWABLE LAXATIVE COMPOSITION,” and claims the benefit of priority of U.S. Provisional Application No. 61/620,688, filed Apr. 5, 2012, entitled “A SOLID, EDIBLE, CHEWABLE LAXATIVE COMPOSITION,” which applications are herein incorporated by reference in their entirety.

### BACKGROUND

Polyethylene glycol (PEG), particularly PEG 3350 is a known osmotic laxative. PEG 3350 is sold in US in laxative compositions which typically are to be combined with an aqueous medium. For example 17 g of PEG powder is dissolved in about 8 ounces of aqueous beverage and consumed to provide a laxative dosage. Examples of such products include Miralax® (MSD consumer care) and Gavilax® (Gavis Pharmaceuticals, NJ).

Formulation of PEG as a beverage or in an aqueous medium is inconvenient, unsatisfying and uncomfortable to the consumer. The aqueous formulation has an undesirable mouth-feel and can create a bloated, overwhelmed feeling in the consumer's stomach. The aqueous formulation is also not a drink that can be discretely consumed so that consumers typically use restrooms in which to mix and drink the formulation. This practice is uncomfortable, disagreeable and ill tasting. The discomfort and inconvenience leads to reduction of dose compliance of the product as directed. Thus, there is a need for more conveniently dosed polyethylene glycol.

While there are certain products, for example Movicol® (Norgine Ltd, Middx UB9 6NS, UK) that are sachets for combination with aqueous media, they contain in addition to polyethylene glycol, electrolytes such as sodium chloride, sodium bicarbonate and potassium chloride. These salts do not impart good taste and suffer from the same discomfort and inconvenience as an aqueous formulation containing only PEG.

Other laxatives formulated for oral consumption suffer from the same problems. All have disagreeable tastes, cause a bloated feeling in the stomach and require inconvenient mixing as aqueous formulations. Chief among these are the laxative preparations with water insoluble substances such as psyllium, bran husk, microcrystalline cellulose and the like. These all form gritty distasteful aqueous formulations that leave thick residue in the glass when drunk.

Therefore, there is a need to provide a laxative dosage form that has a good taste and would enhance patient compliance.

### SUMMARY OF INVENTION

The present invention is directed to a laxative based edible, solid composition. An aspect of the invention provides that the composition preferably is formed as a bar or candy. According to another aspect of the invention, the composition of such ingredients as a laxative, a bulking agent and an optional sweetening agent.

An embodiment of the invention provides that the solid composition is formed as an edible solid bar or candy by heating the ingredients to form a fluid dispersion and cooling the fluid dispersion, preferably in a mold to form a solid bar. In particular, and preferably, another aspect of the invention provides that the laxative is first melted or otherwise converted into a flowable viscous to fluid state. The additional ingredients are then combined with the fluid bulking agent and mixed to form the hot fluid dispersion. The fluid dispersion is added to molds preferably sized to produce mouth-sized pieces, and allowed to cool to ambient temperature.

According to yet another aspect of the invention, the dispersion constituting the chewable, edible composition is solid at ambient temperature.

The edible solid composition formed in this manner has a smooth, non-crystalline, non-gritty mouth feel. Its physical consistency is that of a substantially homogeneous dispersion or preferably a solid solution. The laxative, bulking agent and sweetening agent are physically compatible so that they will form smooth intimate mixtures and will not separate or individually crystallize out of the mixture or solution after formation. Additional components of the composition are also mutually compatible so that separation and/or crystallization do not occur.

Additional aspects of the invention provide that the composition may optionally include electrolytes such as sodium chloride, sodium bicarbonate and potassium chloride. Other optional aspects include sweeteners and flavoring agents that can also be added to the composition as appropriate. Specialized flavors that target saltiness can be also advantageously utilized to reduce the salty taste according to further aspects of the invention. Optionally soluble fibers such as wheat dextrin (for example, Nutriose®, Roquette, France), Inulin, partially hydrolyzed guar gum can be included in the bar.

A further aspect of the invention is the benefit provided by the process employed to produce the solid composition. The melt process provides a better taste and mouth-feel for the solid composition compared with processes for formation such as compression, mixing with adherent substances, mixing with liquids that are evaporated upon standing and the like.

### DETAILED DESCRIPTION

According to the foregoing aspects of the invention, the solid, edible composition may contain such laxatives as polyethylene glycol, castor oil, senna, bisacodyl, sodium pico sulfate, magnesium citrate, magnesium hydroxide (milk of magnesia), magnesium carbonate magnesium sulfate (Epsom salt), sodium sulfate, potassium sulfate, aloes, cascara sacrada, psyllium husk (Metamusil), guar gum, maltodextrins, inulin, docusate sodium, methyl cellulose, monobasic sodium phosphate, dibasic sodium phosphate, glycerin, sorbitol, mannitol, sodium potassium tartrate, mineral oil (liquid paraffin), lactulose, rhubarb, agar agar, figs (Syrup of Figs). L-glucose and any combination thereof. The preferred laxatives are PEG, magnesium citrate, lactulose or bisacodyl and especially preferred laxatives include PEG's of number designations s 400, 3350, 4000, 6000, 18000 and 20000. Of particular preference is PEG 3350.

The bulking agent forming an ingredient aspect of the composition includes carbohydrates and carbohydrate derivatives such as hydrogenated carbohydrates and compounds mimicking carbohydrates either in taste or bulk or pharmaceutical acceptability or any combination thereof. The carbohydrates and carbohydrate derivative and mimics are selected to provide digestive and substance bulk, diluent properties as well as pharmaceutical, nutritional and consumable acceptability. Such bulking agents preferably will have physical properties that will enable melt or fluid processing and will produce firm solids at ambient temperature either as the bulking agents alone or in combination with the sweetening agent and the laxative. The bulking agents include all forms of carbohydrates having the foregoing properties. The bulking agents include but are not limited to such carbohydrates as such as corn syrup, sorbitol, sucrose, dextrose, invert sugar, ludiflash, chocolate, maltitol, high fructose syrup, golden syrup, rice syrup, barley malt syrup, sugar syrup, glycerin, maltodextrins, starch hydrolysate, honey, molasses, lecithin, condensed milk, sweetened condensed milk and any combination thereof. Many of the bulking agents also provide sweetness to the solid composition which minimizes or eliminates the inclusion of separate sweetening agents.

Corn Syrup, Maltitol Syrup and/or maltodextrin are preferred. Sweetening agents that give pleasant mouth feel such as sorbitol can also be utilized. Alternatively other sugar alcohols or carbohydrates can be used such as sugar syrup, mannitol, sorbitol, xylitol, lactitol, isomalt, maltitol, or hydrogenated starch hydrolysates.

Sweetening agents include all manner and configuration of natural and artificial agents that provide a mouth-feel and taste like that of sugar. Included are sugar, lactose, sorbitol, saccharine, aspartame, acesulfame potassium, sucralose, isomalt, mannitol, stevia, cyclamate, dextrose, glucose, fructose, honey, high fructose syrup and any combination thereof. The natural sweetening agents such as sorbitol, dextrose, glucose, sucrose can also be employed in part to provide bulk and diluent properties to the composition.

Artificial sweetening agents such as sucralose, acesulfame, isomalt, mannitol, stevia, Cyclamates, aspartame, saccharine, sugar and flavoring agents are preferred for some purposes especially when dietary constraints are important. Several types of chocolates are available such as dark chocolate, milk chocolate, semi-sweet chocolate, sweet chocolate, hard chocolate, soft chocolate, bitter chocolate, Cocoa mass, cocoa butter mix, hard boiled toffees, and the like. The chocolate can be added as a coat to the bar or included in the bar.

In certain embodiments, less or no corn syrup can be utilized to reduce the calorific value of the bar. Similarly, in certain other embodiments, less or no sorbitol or other sugar alcohols were utilized to reduce the calorific value of the bar. Other embodiments include bars made without bodying or sweetening agents with only chocolate included in the bar. Another embodiment include bars using Soy lecithin and condensed milk in the bar.

Flavoring agents such as bubble gum, cherry, mixed berry, fruit punch, pine apple, caramel, mixed fruit, chocolate, spearmint, peppermint, menthol, licorice, orange, lemon, Watermelon, melagawine, cranberry, tutti frutti, mix of cardamom and orange, vanilla, ice cream soda, salt masking flavor and any combination thereof may be included as optional ingredients.

Electrolytes such as sodium chloride, potassium chloride, potassium bicarbonate, magnesium chloride may also be optionally included.

The compositions of the invention may contain a reasonable portion of the bulking agent, typically at least about 10% by weight, preferably at least about 20% by weight and especially preferably at least about 30 to 40% by weight. The combination of the bulking agent and the natural sweetening agents may be formulated as a combination portion of the composition such as from about 10% to about 80% by weight. The flavoring agents, electrolytes and other optional ingredients may be omitted but when formulated into the composition, may constitute from about 1% up to about 20% by weight of the composition. The laxative can be formulated into the composition according to the FDA guidelines. Up to about 70% of the composition may be a PEG laxative. More potent laxatives such as magnesium citrate, docusate sodium, sodium pico sulfate may be present in lesser weight percents according to the FDA monograph on laxatives.

If caloric intake is to be considered, the carbohydrate quantity provided by the bulking and optional sweetening agents may be kept at a minimum or non-caloric semisynthetic food grade carbohydrate substitutes may be employed. Such non-caloric bulking and sweetening agents include xylitol, mannitol, sorbitol, sucralose, stevia, aspartame, neotame, cyclamate, saccharine, and other non or minimal caloric bulking agents and/or sweeteners such as hydroxyethyl starch, inulin and the like.

According to the inventive aspects described above, the solid, edible compositions of the invention are prepared by melt forming. The bulking agent, PEG or combination there off are heated either to melt it or to convert it into a flowable fluid. The additional ingredients are added while stirring and maintaining the mixture in a melted or flowable fluid condition. After all ingredients are combined, the hot mixture is agitated until a substantially homogeneous suspension or preferably a solution of the ingredients is achieved. The suspension or solution is then placed or poured into receptacles such as molds and allowed to cool to ambient temperature. The molds are shaped to provide appropriately sized pieces of the solid composition suitable for entry into the mouth. The weight percents of ingredients are calculated so as to provide the desired concentration and amount of laxative, sweetening agent, flavoring agent and bulking agent in each piece of chewable, edible, solid composition. The ratios of ingredients can be adjusted to provide pieces of composition that are and remain solid at ambient temperature. The ratios of ingredients per piece of composition are also calculated to deliver the appropriate amount of laxative for the purpose intended. For example, use of the composition to foster natural bowel movements will employ a lesser amount of laxative than will use of the composition for colonic purposes or for complete flushing of the colon in preparation for a full colonoscopy.

It has been found that the foregoing process provides an unexpected benefit to the solid composition of the invention in that its taste and mouth-feel are better than those characteristics of products of the same ingredients prepared by other processes such as compression forming, inclusion of adhering (adhesive) ingredients, molding and covering with edible coating, and mixing while warming but not melting. The compositions of the invention prepared by the foregoing process have little or no waxy taste, little or no gritty mouth-feel and are pleasing to the consumer. In contrast, products produced by alternative methods are waxy in taste and mouth-feel and are non-homogeneous in taste. Chocolate has been found to be a preferred bulking/sweetening agent with low caloric contribution and significant benefit for taste and mouth feel. The chocolate may be dark chocolate or milk chocolate and the milk ingredient may be dairy, coconut, soybean, almond or other milk-like substance. In another embodiment lecithin, condensed milk, sweetened condensed milk, evaporated milk, fat free sweetened condensed milk or the combination there off can be also used as bulking agent to obtain low calorific, non-chocolate product that is homogeneous with pleasant taste.

All publications, patents, and patent applications mentioned in this specification are herein incorporated by reference to the same extent as if each individual publication, patent, or patent application was specifically and individually indicated to be incorporated by reference.

### EXAMPLES

The following experiments exemplify the invention. The process for formation is provided in each example after the tabular description of the ingredients and their amounts. The amounts are given in weight percents relative to the total weight of the composition.

### Example 1

| Ingredients | Amount |
|---|---|
| PEG 3350 | 17 |
| Corn Syrup | 8 |
| (Karo) | |
| Sorbitol 70% | 1 |
| Sucralose | 0.07 |
| Strawberry | 0.2 |
| flavor | |
| Total | 26.07 |

1) Mix Sorbitol 70% and corn syrup together.
2) Heat them in a water bath up to 70 C.
3) Add PEG 3350 and mix them together. Dissolve Sucralose in approximately 2 ml of water and add to the mixture.
4) Cool the mixture to 50-55 C and add strawberry flavor.
   Pour the mixture into a mold and cool it. Once the mold is set, cut the bars into appropriate size.

### Example 2

| Ingredients | Amount |
|---|---|
| PEG 3350 | 17 |
| Corn Syrup | 7 |
| (Karo-light) | |
| Sorbitol 70% | 5.57 |
| Sucralose | 0.08 |
| Citric acid | 0.15 |
| Red #40 | 0.05 |
| Strawberry | 0.15 |
| flavor | |
| Total | 29.9 |

1) Melt PEG 3350 on a water bath. When the temperature reads 70 C, add corn syrup and sorbitol 70% and mix
2) Add sucralose and mix
3) Add citric acid and dissolve
4) Continue heating at about 70 C for 15 minutes
5) Cool the mixture under stirring
6) At or below 50 C, add color and flavor
7) Pour into a mold
8) Cut into pieces when the mold is set

### Example 3

| Ingredients | Amount |
|---|---|
| PEG 3350 | 17 |
| Corn Syrup | 7 |
| (Karo-light) | |
| Sorbitol 70% | 4.875 |
| Sucralose | 0.05 |
| Condensed | 5 |
| milk | |
| Pineapple | 0.05 |
| flavor | |
| Tropical fruit | 0.025 |
| flavor | |
| Total | 34 |

1) Melt PEG 3350 on a water bath and heat on a hot plate. When the temperature reads 70 C, add corn syrup and sorbitol 70% and mix.
2) Add condensed milk, sucralose and mix.
3) Continue heating at about 70 C for 15 minutes
4) Cool the mixture under stirring
5) Between 50-55 C, add color and flavor.
6) Pour into a mold.
7) Cut into pieces when the mold is set.
   Observation: The product did not set. Very soft.

### Example 4

| Ingredients | Amount |
|---|---|
| PEG 3350 | 17 |
| Corn Syrup | 7 |
| Sorbitol 70% | 8 |
| Sucralose | 0.05 |
| Mixed Berry | 0.1 |
| flavor | |
| Total | 32 |

1) Mix corn syrup and sorbitol 70% and heat on water bath to 60 C
2) Add PEG 3350 and mix well.
3) Add sucralose and mix.
4) Continue heating for 30 minutes.
5) Cool the mixture under stirring.
6) Between 50-55 C, add color and flavor.
7) Pour into a mold.
8) Cut into pieces when the mold is et. Observation: Taste is very good.

### Example 5

| Ingredients | Amount |
|---|---|
| PEG 3350 | 17 |
| Corn Syrup | 8 |
| Sorbitol 70% | 8 |
| Sucralose | 0.05 |
| Strawberry | 0.1 |
| flavor | |
| Total | 33 |

1) Mix corn syrup and sorbitol 70% and heat on water bath to 70 C
2) Add PEG 3350 and mix well.
3) Add sucralose and mix.
4) Continue heating for 30 minutes.
5) Cool the mixture under stirring.
6) At 50-55 C, add color and flavor.
7) Pour into a mold.
8) Cut into pieces when the mold is set.

### Example 6

| Ingredients | Amount |
|---|---|
| PEG 3350 | 17 |
| Corn Syrup | 10 |
| Sorbitol 70% | 7 |
| Sacralose | 0.041 |
| Mint flavor | 0.12 |
| Total | 35 |
| Chocolate | ~5 |
| coat | |

1) Mix corn syrup and sorbitol 70% and heat on water bath to 70 C
2) Add PEG 3350 and mix well.
3) Add sucralose and mix.
4) Continue heating for 30 minutes.
5) Cool the mixture under stirring.
6) At 50-55 C, add flavor.
7) Pour into a mold.
8) Cut into pieces when the mold is set.
9) Coat with melted chocolate.

### Example 7

| Ingredients | Amount |
|---|---|
| PEG 3350 | 17 |
| Corn Syrup | 10 |
| Sorbitol 70% | 4 |
| Sucralose | 0.041 |
| Strawberry | 0.1 |
| flavor | |
| Red color | 0.0005 |
| Total | 31 |
| Chocolate | ~5 |
| coat | |

1) Mix corn syrup and sorbitol 70% and heat on water bath to 70 C
2) Add PEG 3350 and mix well.
3) Add sucralose and mix.
4) Continue heating for 30 minutes.
5) Cool the mixture under stirring.
6) At 50-55 C, add color and flavor.
7) Pour into a mold.
8) Cut into pieces when the mold is set.
9) Coat with melted chocolate.

### Example 8

| Ingredients | Amount |
|---|---|
| PEG 3350 | 17 |
| Ludiflash ®* | 13 |
| Sucralose | 0.041 |
| Orange flavor | 0.1 |
| Mg Stearate | 0.015 |
| Total | 30 |

*BASF

1) Mix PEG 3350, Ludiflash, Sucralose and Orange flavor
2) Add Mg stearate and mix
3) Compress using a tablet machine
   Observation: Not as good as the bar.

### Example 9

| Ingredients | Amount |
|---|---|
| PEG 3350 | 17 |
| Corn Syrup | 5 |
| Sorbitol 70% | 3 |
| Sucralose | 0.041 |
| Mint flavor | 0.1 |
| Total | 25 |

1) Mix corn syrup and sorbitol 70% and heat on water bath to 70 C
2) Add PEG 3350 and mix well.
3) Add sucralose and mix.
4) Continue heating for 30 minutes.
5) Cool the mixture under stirring.
6) At 50-55 C, add color and flavor.
7) Pour into a mold.
8) Cut into pieces when the mold is set.
   Observation: The bar did not set well. Sticky bar. Taste is good.

### Example 10

| Ingredients | Amount |
|---|---|
| PEG 3350 | 17 |
| Corn Syrup | 10 |
| Sucralose | 0.041 |
| Mint flavor | 0.1 |
| Total | 25 |

1) Mix corn syrup and PEG 3350 and heat on water bath to 70 C
2) Add sucralose and mix.
3) Continue heating for 30 minutes.
4) Cool the mixture under stirring.
5) At 50-55 C, add color and flavor.
6) Pour into a mold.
7) Cut into pieces when the mold is set.

### Example 11

| Ingredients | Amount |
|---|---|
| PEG 3350 | 17 |
| Corn Syrup | 10 |
| Sorbitol 70% | 4 |
| Sucralose | 0.041 |
| Sodium | 0.351 |
| Chloride | |
| Sodium | 0.17 |
| bicarbonate | |
| Pot Chloride | 0.047 |
| Mint flavor | 0.1 |
| Total | 31 |

1) Mix corn syrup and sorbitol and heat on water bath to 70 C.
2) Add PEG 3350 and mix.
3) Add sucralose and mix.
4) Dissolve sodium chloride, sodium bicarbonate and Potassium chloride in small quantity of water and add.
5) Continue heating for 30 minutes.
6) Cool the mixture under stirring.
7) At 50-55 C add flavor.
8) Pour into a mold.
   Observation: Did not set well. Became pasty. Salts seem to inhibit proper setting. Salty taste.

### Example 12

| Ingredients | Amount |
|---|---|
| PEG 3350 | 17 |
| Corn Syrup | 7 |
| Sorbitol 70% | 3 |
| Sucralose | 0.041 |
| Mint flavor | 0.1 |
| Total | 27 |

1) Mix corn syrup and sorbitol 70% and heat on water bath to 70 C.
2) Add PEG 3350 and mix well.
3) Add sucralose and mix.
4) Continue heating for 30 minutes.
5) Cool the mixture under stirring.
6) At 50-55 C, add color and flavor.
7) Pour into a mold.
8) Cut into pieces when the mold is set.

### Observation: Sticky

### Example 13

| Ingredients | Amount |
|---|---|
| PEG 3350 | 17 |
| Corn Syrup | 10 |
| Sorbitol 70% | 2 |
| Sucralose | 0.041 |
| Mint flavor | 0.1 |
| Total | 29 |

1) Mix corn syrup and sorbitol 70% and heat on water bath to 70 C
2) Add PEG 3350 and mix well.
3) Add sucralose and mix.
4) Continue heating for 30 minutes.
5) Cool the mixture under stirring.
6) At 50-55 C, add color and flavor.
7) Pour into a mold.
8) Cut into pieces when the mold is set.

### Example 14

| Ingredients | Amount |
|---|---|
| PEG 3350 | 17 |
| Corn Syrup | 15 |
| Sucralose | 0.041 |
| Sodium | 0.351 |
| Chloride | |
| Sodium | 0.179 |
| bicarbonate | |
| Pot Chloride | 0.047 |
| Mint flavor | 0.1 |
| Total | 32 |

1) Mix corn syrup and sorbitol and heat on water bath to 70 C
2) Add PEG 3350 and mix
3) Add sucralose and mix.
4) Dissolve sodium chloride, sodium bicarbonate and Potassium chloride in small quantity of water and add
5) Continue heating for 30 minutes.
6) Cool the mixture under stirring.
7) At 50-55 C, add flavor.
8) Pour into a mold
   Sticky. Little rubbery.

### Example 15

| Ingredients | Amount |
|---|---|
| PEG 3350 | 17 |
| Corn Syrup | 10 |
| Chocolate - | 8 |
| semisweet* | |
| Sucralose | 0.041 |
| Mint flavor | 0.1 |
| Total | 35 |

*Nestle ™ semi-sweet chocolate (0-28000-21580-4). Ingredients: semisweet chocolate, sugar, chocolate, cocoa butter, milk fat, soy lecithin, natural flavors

1) Mix corn syrup and sorbitol and heat on water bath to 70 C
2) Add PEG 3350 and mix
3) Add chocolate and mix
4) Add sucralose and mix.
5) Continue heating for 30 minutes.
6) Cool the mixture under stirring.
7) At 50-55 C, add flavor.
8) Pour into a mold.
   Observation: Sets well. Good taste & texture.

### Example 16

| Ingredients | Amount |
|---|---|
| PEG 3350 | 17 |
| Corn Syrup | 15 |
| Chocolate - | 8 |
| semisweet* | |
| Sucralose | 0.041 |
| Sodium | 0.351 |
| Chloride | |
| Sodium | 0.179 |
| bicarbonate | |
| Pot Chloride | 0.047 |
| Mint flavor | 0.1 |
| Total | 40 |

*Nestle ™ semi-sweet chocolate (0-28000-21580-4). Ingredients: semisweet chocolate, sugar, chocolate, cocoa butter, milk fat, soy lecithin, natural flavors

1) Mix corn syrup and sorbitol and heat on water bath to 70 C.
2) Add PEG 3350 and mix.
3) Add chocolate and mix.
4) Add sucralose and mix.
5) Cool to 50-55 C or less and add the water solution of salts.
6) Continue heating for 30 minutes.
7) Cool the mixture under stirring.
8) At 50-55 C, add flavor.
9) Pour into a nmold
   Little rubbery. Taste is good.

### Example 17

| Ingredients | Amount |
|---|---|
| PEG 3350 | 17 |
| Corn Syrup | 15 |
| Chocolate - | 8 |
| Dark | |
| Ghirardelli | |
| chocolate* | |
| Sucralose | 0.041 |
| Sodium | 0.351 |
| Chloride | |
| Sodium | 0.179 |
| bicarbonate | |
| Pot Chloride | 0.047 |
| Mint flavor | 0.1 |
| Total | 40 |

*Ghirardelli unsweetened chocolate (7-47599-60106-4); Ingredients: Unsweetened chocolate.

1) Mix corn syrup and sorbitol and heat on water bath to 70 C.
2) Add PEG 3350 and mix.
3) Add chocolate and mix.
4) Add sucralose and mix.
5) Cool to 50 C or less and add the salts.
6) Continue heating for 30 minutes.
7) Cool the mixture under stirring.
8) At 50-55 C add flavor.
9) Pour into a mold.
Good taste and texture.

### Example 18

| Ingredients | Amount |
|---|---|
| PEG 3350 | 13.125 |
| Dark | 6.18 |
| Chocolate* | |
| Sucralose | 0.041 |
| Sodium | 0.351 |
| Chloride | |
| Sodium | 0.179 |
| bicarbonate | |
| Pot Chloride | 0.047 |
| Mint flavor | 0.1 |
| Total | 20 |

*ADM cocoa: DC-1015 value BS Choc. Ingredients: Sugar, chocolate liquor, cocoa butter, chocolate liquor processed with alkali, soy lecithin, vanilla, salt and artificial flavor. May contain milk.

1) Melt PEG to 65 C on a water bath.
2) Add chocolate and mix.
3) Add sucralose and mix.
4) Cool to below 60 C and add salts and mix.
5) Cool the mixture under stirring.
6) At 50-55 C add flavor.
7) Pour into a mold.

### Example 19

| Ingredients | Amount |
|---|---|
| PEG 3350 | 13.125 |
| Milk | 6.18 |
| Chocolate* | |
| Sucralose | 0.041 |
| Sodium | 0.351 |
| Chloride | |
| Sodium | 0.179 |
| bicarbonate | |
| Pot Chloride | 0.047 |
| Mint flavor | 0.1 |
| Total | 20 |

*ADM MC-050 Marquis MC. Ingredients: Sugar, milk, cocoa butter, chocolate liquor, soy lecithin, and vanilla.

The processing procedure used is the same as described for experiment 18.

### Example 20

| Ingredients | Amount |
|---|---|
| PEG 3350 | 13.125 |
| Corn Syrup | 11.7 |
| Dark | 6.18 |
| Chocolate | |
| (ADM DC- | |
| 1015) | |
| Sucralose | 0.041 |
| Sodium | 0.351 |
| Chloride | |
| Sodium | 0.179 |
| bicarbonate | |
| Pot Chloride | 0.047 |
| Mint flavor | 0.1 |
| Total | 31 |

1) Melt PEG to 65 C on a water bath. Add corn syrup and mix.
2) Add chocolate and mix.
3) Add sucralose and mix.
4) Cool to below 60 C and add salts and mix.
5) Cool the mixture under stirring.
6) At 50-55 C add flavor.
7) Pour into a mold.

### Example 21

| Ingredients | Amount |
|---|---|
| PEG 3350 | 13.125 |
| Corn Syrup | 11.7 |
| Milk | 6.18 |
| Chocolate | |
| (MC-050) | |
| Sucralose | 0.041 |
| Sodium | 0.351 |
| Chloride | |
| Sodium | 0.179 |
| bicarbonate | |
| Pot Chloride | 0.047 |
| Mint flavor | 0.1 |
| Total | 31 |

The processing procedure used is the same as described for experiment 20.

### Example 22

| Ingredients | Amount |
|---|---|
| PEG 3350 | 13.125 |
| Dark | 8.333 |
| Chocolate | |
| (ADM DC- | |
| 1015) | |
| Sucralose | 0.041 |
| Sodium | 0.351 |
| Chloride | |
| Sodium | 0.179 |
| bicarbonate | |
| Pot Chloride | 0.047 |
| Mint flavor | 0.1 |
| Total | 22 |

The processing procedure used is conducted as describe for experiment 20.

### Example 23

| Ingredients | Amount |
|---|---|
| PEG 3350 | 17 |
| Corn Syrup | 12 |
| Sucralose | 0.041 |
| Mint flavor | 0.1 |
| Total | 29.14 |

1) Melt PEG to 70 C on a water bath. Add corn syrup and mix.
2) Add sucralose and mix.
3) At about 65 C add flavor.
4) Pour into a mold.

### Example 24

| Ingredients | Amount |
|---|---|
| PEG 3350 | 17 |
| Corn Syrup | 12 |
| Sucralose | 0.041 |
| Strawberry | 0.1 |
| flavor | |
| Red #40 | 0.05 |
| Total | 29.14 |

The processing procedure used is similar to that of experiment 23.

### Example 25

| Ingredients | Amount |
|---|---|
| PEG 3350 | 17 |
| Dark | 12 |
| chocolate | |
| (ADM DC | |
| 1015) | |
| Sucralose | 0.041 |
| Mint flavor | 0.1 |
| Total | 29.14 |

The processing procedure used is similar to that of experiment 23.

### Example 26

| Ingredients | Amount |
|---|---|
| PEG 3350 | 17 |
| Dark | 12 |
| chocolate | |
| (ADM DC | |
| 1015) | |
| Sucralose | 0.041 |
| Strawberry | 0.1 |
| flavor | |
| Total | 29.14 |

The processing procedure used is similar to that of experiment 23.

### Example 27

| Ingredients | Amount |
|---|---|
| PEG 3350 | 13.125 |
| Dark | 9.0 |
| Chocolate | |
| (ADM DC- | |
| 1015) | |
| Sucralose | 0.041 |
| Sodium | 0.351 |
| Chloride | |
| Sodium | 0.179 |
| bicarbonate | |
| Pot Chloride | 0.047 |
| Mint flavor | 0.1 |
| Total | 22 |

The processing procedure used is similar to that of experiment 20.

### Example 28

| Ingredients | Amount |
|---|---|
| PEG 3350 | 13.125 |
| Dark | 9.0 |
| Chocolate | |
| (ADM DC- | |
| 1015) | |
| Sucralose | 0.041 |
| Sodium | 0.351 |
| Chloride | |
| Sodium | 0.179 |
| bicarbonate | |
| Pot Chloride | 0.047 |
| Strawberry | 0.1 |
| flavor | |
| Total | 22 |

The processing procedure used is similar to that of experiment 20.

### Example 29

| Ingredients | Amount |
|---|---|
| PEG 3350 | 13.125 |
| Corn Syrup | 15 |
| Sucralose | 0.041 |
| Sodium | 0.351 |
| Chloride | |
| Sodium | 0.179 |
| bicarbonate | |
| Pot Chloride | 0.047 |
| Strawberry | 0.1 |
| flavor | |
| Total | 28 |

The processing procedure used is similar to that of experiment 20.

### Example 30

| Ingredients | Amount |
|---|---|
| PEG 3350 | 13.125 |
| Corn Syrup | 15 |
| Sucralose | 0.041 |
| Sodium | 0.351 |
| Chloride | |
| Sodium | 0.179 |
| bicarbonate | |
| Pot Chloride | 0.047 |
| Strawberry | 0.1 |
| flavor | |
| Red #40 | 0.05 |
| Total | 28 |

The processing procedure used is similar that of experiment 20.

### Example 31

| Ingredients | Amount |
|---|---|
| PEG 3350 | 17 |
| Bisacodyl | 0.005 |
| Dark | 12 |
| Chocolate | |
| ADM DC- | |
| 1015 | |
| Sucralose | 0.041 |
| Mint flavor | 0.1 |
| Total | 29 |

The processing procedure used is similar as that of experiment 20.

### Example 32

| Ingredients | Amount |
|---|---|
| PEG 3350 | 17 |
| Sod Pico | 0.005 |
| Sulfate | |
| Dark | 12 |
| Chocolate | |
| ADM DC- | |
| 1015 | |
| Sucralose | 0.041 |
| Mint flavor | 0.1 |
| Total | 29 |

The processing procedure used is similar as that of experiment 20.

### Example 33

| Ingredients | Amount |
|---|---|
| PEG 3350 | 17 |
| Dark | 12 |
| Chocolate | |
| ADM DC- | |
| 1015 | |
| Sucralose | 0.041 |
| Mint flavor | 0.1 |
| Total | 29 |

The processing procedure used is similar as that of experiment 20.

### Example 34

| Ingredients | Amount |
|---|---|
| PEG 3350 | 17 |
| Docusate | 0.1 |
| Sodium | |
| Dark | 12 |
| Chocolate | |
| ADM DC- | |
| 1015 | |
| Sucralose | 0.041 |
| Mint flavor | 0.1 |
| Total | 29 |

The processing procedure used is similar as that of experiment 20.

### Example 35

| Ingredients | Amount |
|---|---|
| PEG 3350 | 17 |
| White | 12 |
| Chocolate | |
| Sucralose | 0.041 |
| Mint flavor | 0.1 |
| Total | 29 |

The processing procedure used is similar as that of experiment 20.

### Formulation Summary (Per Bar in Grams)

| Item | Ingredients | RB1 | RB115002 | RB115003 | RB115004 | RB115005 | RB115006 | RB115007 |
|---|---|---|---|---|---|---|---|---|
| 1 | PEG 3350 | 17.0 | 17.0 | 17.0 | 17.0 | 17.0 | 17.0 | 17.0 |
| 2 | Corn Syrup | 8. | 7.0 | 7.0 | 7.0 | 8.0 | 10.0 | 10.0 |
| 3 | Sorbitol 70% | 1. | 5.5 | 4.88 | 8.0 | 8.0 | 7.0 | 4.0 |
| 4 | Sucralose | 0.07 | 0.08 | 0.05 | 0.05 | 0.05 | 0.04 | 0.04 |
| 5 | Chocolate | | | | | | 5.0 | 5.0 |
| | | | | | | | coat | coat |
| 6 | Mint flavor | | | | | | 0.12 | |
| 7 | Strawberry Flavor | 0.2 | 0.15 | | | | | |
| 8 | Pine app. Fla | | | 0.05 | | | | |
| 9 | Mixed berry | | | | 0.1 | | | |
| 10 | Trop. fla | | | 0.03 | | | | |
| 11 | Orange fla | | | | | | | |
| 12 | Citric acid | | 0.15 | | | | | |
| 13 | Ludiflash | | | | | | | |
| 14 | Mag. Stear | | | | | | | |
| 15 | Red # 40 | | 0.05 mg | | | | | 0.01 |
| 16 | Condensed milk | | | 5.0 | | | | |
| Total Wt | | 26.27 | 29.9 | 34.0 | 32.1 | 33.1 | 40.0 | 37.0 |
| Comments | | Exploratory | Waxy | Very soft | Very soft | Good | Good | Good |
| | | Waxy taste | | | Taste ok | | | Chocolate |
| | | More sweet | | | | | | coat |

### Formulation Summary (Per Tablet Equivalent)

### Tablet Dosage Form

| | | RB | RB | RB |
|---|---|---|---|---|
| Item # | Ingredients | 115008 | 115009 | 115010 |
| 1 | PEG 3350 | 17.0 | 17.0 | 17.00 |
| 2 | Sucralose | | 0.05 | 0.041 |
| 3 | Orange | 0.13 | 0.13 | 0.100 |
| | flavor | | | |
| 4 | Ludiflash | | 5.00 | 13.000 |
| 5 | Mag. Stear | | | 0.0150 |
| Total Wt | | 17.13 | 22.18 | 30.156 |
| Comments | | Tablets | Tablets | Tablets |
| | | compressed by | compressed by | compressed |
| | | manual | manual | with Mini- |
| | | Hard | operation | press Not |
| | | | Hard | as good |
| | | | | as bar |

### Formulation Summary with PEG+Electrolytes (Per Bar in Grams)

| Item | Ingredient. | RB115011 | RB115012 | RB1150013 | RB115014 | RB115015 | RB115016 | RB115017 | RB115018 | RB115019 |
|---|---|---|---|---|---|---|---|---|---|---|
| 1 | PEG 3350 | 17.0 | 17.0 | 17.0 | 17.0 | 17.0 | 17.0 | 17.0 | 17.0 | 17.0 |
| 2 | Corn Syrup | 5.0 | 10.0 | 10.0 | 7.0 | 10.0 | 15.0 | 10.0 | 15.0 | 15.0 |
| 3 | Sorbitol 70% | 3.0 | | 4.0 | 3.0 | 2.0 | | | | |
| 4 | Sucralose | 0.04 | 0.04 | 0.04 | 0.04 | 0.04 | 0.04 | 0.04 | 0.04 | 0.04 |
| 5 | Chocolate | | | | | | | 8.0 | 8.0 | 8.0 |
| | | | | | | | | | Semi | Dark |
| | | | | | | | | | Sweet | Chiredelli |
| 6 | Sodium Chloride | | | 0.35 | | | 0.35 | | 0.35 | 0.35 |
| 7 | Sod. Bi carb | | | 0.17 | | | 0.17 | | 0.17 | 0.17 |
| 8 | Pot. Chloride | | | 0.04 | | | 0.04 | | 0.04 | 0.04 |
| 9 | Mint flavor | 0.1 | 0.1 | 0.1 | 0.1 | 0.1 | 0.1 | 0.1 | 0.1 | 0.1 |
| Total Wt | | 25.1 | 27.1 | 31.1 | 27.1 | 29.1 | 32.7 | 35.1 | 40.7 | 40.7 |
| Comments | | Poor | Good | Do not | Sticky | Sticky | Less | Good | Good | Good |
| | | Set | Set | Set | Rubbery | | sticky | Set | set | set |
| | | Taste | Taste | Salt | | | Taste | Good | Taste | Taste |
| | | ok | ok | affects | | | ok | taste | good | good |
| | | | | setting | | | | | | |

### Formulation Summary with Electrolytes (Per Bar in Grams)

| Item | Ingredients | RB115020 | RB115021 | RB1150022 | RB115023 | RB115025 |
|---|---|---|---|---|---|---|
| 1 | PEG 3350 | 13.125 | 13.125 | 13.125 | 13.125 | 13.125 |
| 2 | Corn Syrup | | | 11.700 | 11.700 | |
| 3 | Sucralose | 0.041 | 0.041 | 0.041 | 0.041 | 0.041 |
| 4 | Chocolate | 6.180 | 6.180 | 6.180 | 6.180 | 8.333 |
| | | Dark | Milk | Dark | Milk | Dark |
| 5 | Sodium Chloride | 0.351 | 0.351 | 0.351 | 0.351 | 0.351 |
| 6 | Sod. Bi carb | 0.179 | 0.179 | 0.179 | 0.179 | 0.179 |
| 7 | Pot. Chloride | 0.047 | 0.047 | 0.047 | 0.047 | 0.047 |
| 8 | Mint flavor | 0.100 | 0.100 | 0.100 | 0.100 | 0.100 |
| Total Wt | | 20.0 | 20.0 | 31.72 | 31.72 | 22.4 |
| Comments | | V. Good Set | Good Set | Harder chocolate | Sticky | Taste good |
| | | Release from | A little sticky | Release form | Taste | Easy release |
| | | mold easily | Release from | mold easily | ok | from mold |
| | | | mold is bad | Taste good | | A lead for future |
| | | | | | | experiments |

### Formulation Summary (Per Bar—in Grams)

| | | RB | RB | | RB | |
|---|---|---|---|---|---|---|
| Item | Ingredients | 115026 | 115027 | | 115028 | RB 1 |
| 1 | PEG 3350 | 17.000 | 17.00 | | 17.00 | 1 |
| 2 | Corn Syrup | 12.000 | 12.00 | | | |
| 3 | Sucralose | 0.041 | 0.041 | | 0.041 | 0. |
| 4 | Chocolate | | | | 12.00 | 1 |
| 5 | Mint flavor | 0.100 | | | 0.100 | |
| 6 | Strawberry | | 0.100 | | | 0. |
| | flavor | | | | | |
| 7 | Red # 40 | | 0.05 | mg | | |
| Total Wt | | 29.141 | 29.141 | | 29.141 | 29.141 |

| | | | | |
|---|---|---|---|---|
| Comments | Difficulty | Difficulty | Good | Good |
| | in | in | taste; | taste; |
| | releasing | releasing | release | release |
| | from | from mold | from | from |
| | mold | | mold | mold |
| | | | easily | easily |

### Bar Formulation Summary with PEG+Electrolytes (Per Bar—in Grams)

| | | RB | RB | RB | RB | |
|---|---|---|---|---|---|---|
| Item | Ingredients | 115030 | 115031 | 115032 | 115033 | |
| 1 | PEG 3350 | 13.125 | 13.125 | 13.125 | 13.125 | |
| 2 | Corn Syrup | | | 15.000 | 15.000 | |
| 3 | Sucralose | 0.041 | 0.041 | 0.041 | 0.041 | |
| 4 | Chocolate | 9.000 | 9.000 | | | |
| 5 | Sodium | 0.351 | 0.351 | 0.351 | 0.351 | |
| | Chloride | | | | | |
| 6 | Sod. Bi carb | 0.179 | 0.179 | 0.179 | 0.179 | |
| 7 | Pot. Chloride | 0.049 | 0.049 | 0.049 | 0.049 | |
| 8 | Mint flavor | 0.100 | | 0.100 | | |
| 9 | Strawberry | | 0.100 | | 0.100 | |
| | Flavor | | | | | |
| 10 | Red # 40 | | | | 0.05 | mg |
| | Total Wt | 22.845 | 22.845 | 28.845 | 28.845 | |

| | | | | |
|---|---|---|---|---|
| Comments | Good | Good | Difficult | Difficult to |
| | taste; | taste; | to | release from |
| | Good | Good | release | mold |
| | release | release | from | |
| | from | from | mold | |
| | mold | mold | | |

### Formulation Summary (Per Bar in Grams)

| Item | Ingredients | RB115034 | RB115035 | RB115036 | RB115037 | RB115038 |
|---|---|---|---|---|---|---|
| 1 | PEG 3350 | 17 | 17 | 17 | 17 | 17 |
| 2 | Bisacodyl | 0.005 | | | | |
| 3 | Sod Pico Sulfate | | 0.005 | | | |
| 4 | Docusate Sodium | | | | 0.1 | |
| 5 | Dark Chocolate | 12 | 12 | 12 | 12 | |
| 6 | White Chocolate | | | | | 12 |
| 7 | Sucralose | 0.041 | 0.041 | 0.041 | 0.041 | 0.041 |
| 8 | Mint flavor | 0.100 | 0.100 | 0.100 | 0.1 | 0.1 |
| | Total Wt | 29 | 29 | 29 | 29 | 29 |

### Formulation Summary (Per Bar in Grams)

| Item | Ingredients | RB-115039 | RB-115040 | RB-115041 |
|---|---|---|---|---|
| 1 | PEG 3350 | — | 4.000 | 4.000 |
| 2 | Pectin | 0.320 | 0.320 | 0.320 |
| 3 | Corn Syrup | 4.000 | 4.000 | 4.000 |
| 4 | Sugar | 3.200 | 3.280 | 3.280 |
| 5 | Citric acid | 0.128 | 0.128 | 0.128 |
| 6 | Strawberry fla | 0.010 | 0.010 | 0.020 |
| 7 | Purified water | 2.280 | 2.280 | 2.880 |
| Comments | | Good | Good taste | Good taste |
| | | taste | A little | A little |
| | | Soft | harder | harder |
| | | | in | in |
| | | | texture | texture |

### Chocolate Bars

| Item | Ingredients | RB 115053 | RB115056 |
|---|---|---|---|
| 1 | PEG 3350 | 17.000 | 17.000 |
| 2 | Bisacodyl | — | — |
| 3 | Sodium Pico | — | — |
| | Sulfate | | |
| 4 | Docusate Sod | — | — |
| 5 | Sucralose | 0.041 | 0.041 |
| 6 | Chocolate | 12.000 | 17.000 |
| | | (white) | (white) |
| 7 | Mint flavor | | |
| 8 | Strawberry | 0.100 | 0.100 |
| | flavor | | |
| 9 | Red # 40 | 0.0004 | 0.005 |
| | Total Wt | 29.1414 | 34.100 |
| | Comments | Good | Not |
| | | taste | good |
| | | Hard | in taste |
| | | texture | and |

### Chocolate Bars

| Item | Ingredients | RB 115068 | RB115069 | RB115072 | RB115073 | RB115075 |
|---|---|---|---|---|---|---|
| 1 | PEG 3350 | 17.000 | 17.000 | 17.000 | 17.000 | 17.000 |
| 2 | Sucralose | 0.041 | 0.041 | 0.041 | 0.041 | 0.041 |
| 3 | Dark Chocolate - ADM | 13.000 | — | 13.000 | — | 13.000 |
| | | | | (ADM) | | (ADM) |
| 4 | White Chocolate | | 13.000 | | 13.000 | — |
| | | | (ADM) | | (Nestle) | |
| 5 | Purified Water | — | — | — | — | 0.416 |
| 6 | Mint flavor | | | | | |
| 7 | Strawberry flavor | 0.100 | 0.100 | 0.100 | 0.100 | 0.100 |
| 8 | Red # 40 | | 0.0005 | | 0.0005 | |
| | Total Wt | 30.141 | 30.142 | 30.141 | 30.142 | 30.557 |
| | Comments | Good | Good | Silicon | Appearance | Appearance |
| | | Taste | Taste | pop mold | is good | is good |
| | | Appearance | Appearance | Good taste | Good taste | Good taste |
| | | not good | not good | Appearance | | Texture is |
| | | | | not good | | good |

Procedure
068: Melt PEG in a container on a water bath at 80° C. Add Sucralose and dissolve. Mix well. Add Chocolate and mix well to disperse uniformly. Start cooling, add flavor. Mix well. Pour into molds. Cool to set.
069: Melt PEG in a container on a water bath at 80° C. Add Sucralose and dissolve. Mix well. Add Chocolate and mix well to disperse uniformly. Add color and mix well. Start cooling, add flavor. Mix well. Pour into molds. Cool to set.
072: Same as 068
073: ame as 069
075: Melt PEG in a container on a water bath at 80° C. Dissolve Sucralose in a small quantity of water and add to the bulk. Mix well. Add Chocolate and mix well to disperse uniformly. Start cooling, add flavor. Mix well. Pour into molds. Cool to set.

### Chocolate Bars

| Item | Ingredients | RB115070 | RB115071 |
|---|---|---|---|
| 1 | PEG 3350 | 17.000 | 17.000 |
| 2 | Sucralose | 0.041 | 0.041 |
| 3 | Sod. Chloride | 0.351 | 0.351 |
| 4 | Pot. Chloride | 0.049 | 0.049 |
| 5 | Sod. bicarbonate | 0.179 | 0.179 |
| 6 | Dark | 16.155 | — |
| | Chocolate - | | |
| | ADM | | |
| 7 | White | — | 116.155 |
| | Chocolate | | |
| | (Nestle) | | |
| 8 | Mint flavor | 0.100 | 0.100 |
| | Total Wt | 30.000 | 30.000 |
| | Comments | Good Taste | Good Taste |
| | | Appearance | Appearance |
| | | not | is |
| | | good | okay |
| | | Texture | Texture is |
| | | is spotty | hard |

Procedure
070 & 071: Melt PEG in a container on a water bath at 80° C. till it forms a clear solution. Add Sucralose and dissolve. Grind all the 3 salts with a mortar and pestle and add to the bulk and mix well to disperse uniformly. Add the chocolate and disperse uniformly maintaining the temperature at 80° C. Add the flavor, mix well .pour into molds and cool to set.

### Chocolate Bar

| | | | | | PEG 3350 + Electrolytes | | |
|---|---|---|---|---|---|---|---|
| | | PEG 3350 alone | | | | | RB115-083A |
| Item | Ingredients | RB115-081 | RBMP-115-88 | RB115-082 | RB115-080 | RB115-083 | Chocolate coated |
| 1 | PEG 3350 | 17.000 | 17.000 | 17.000 | 13.125 | 13.125 | 13.125 |
| 2 | Sucralose | 0.042 | 0.040 | 0.042 | 0.042 | 0.042 | 0.042 |
| 3 | P. Water | 0.832 | 0.830 | 0.832 | 0.832 | 0.832 | 0.832 |
| 4 | Sod. Benzoate | — | 0.030 | — | — | — | |
| 5 | Sodium Chloride | — | — | — | 0.351 | 0.351 | 0.351 |
| 6 | Pot. Chloride | — | — | — | 0.049 | 0.049 | 0.049 |
| 7 | Sod. bi carb | — | — | — | 0.179 | 0.179 | 0.179 |
| 8 | Dark Chocolate (ADM) | 12.000 | 12.000 | | 11.000 | | |
| 9 | White Chocolate | | | 12.000 | | 11.000 | 11.000 |
| | | | | (Nestle) | | (Nestle) | (Nestle) |
| 10 | Mint flavor | — | — | 0.100 | 0.100 | 0.100 | 0.100 |
| 11 | Strawberry Flavour | 0.100 | 0.100 | — | — | — | — |
| | Total Wt | 29.975 | 30.000 | 29.975 | 25.261 | 25.261 | 25.261 + |
| | | | | | | | 5 gm. dark |
| | | | | | | | chocolate coat |
| | Comments | Brown | Brown | White | Brown | White | White core |
| | | | Taste texture | | | | inside brown |
| | | | appearance- | | | | coat outside |
| | | | good | | | | |

Procedure
081: Melt PEG in a container on a water bath at 80° C. till it forms a clear solution. Dissolve Sucralose in a small quantity of water and add to the bulk. Add the chocolate, mix well to distribute uniformly maintaining the temperature at 80° C. Discontonue heating, add the flavor and mix well. Pour into molds and cool to set.
082: Same as 081
088: Melt PEG in a container on a water bath at 80° C. till it forms a clear solution. Dissolve Sodium benzoate and Sucralose in a small quantity of water and add to the bulk. Add the chocolate, mix well to distribute uniformly maintaining the temperature at 80° C. Discontonue heating, add the flavor and mix well. Pour into molds and cool to set.
080: Melt PEG in a container on a water bath at 80° C. till it forms a clear solution. Add Sucralose and dissolve. Grind all the 3 salts with a mortar and pestle and add to the bulk and mix well to disperse uniformly. Add the chocolate and disperse uniformly maintaining the temperature at 80° C. Add the flavor, mix well. Pour into molds and cool to set.
083A: The core chocolates from the experiment 083 are coated with 5 gms of dark chocolate to form a uniform coat.

### Non-Chocolate Bars (Grams Per Bar)

| Item | Ingredients | RB115-091 | RB115-092 | RB115-093 | RB115-094 | RB115-095 |
|---|---|---|---|---|---|---|
| 1 | PEG 3350 | 17.000 | 17.000 | 13.125 | 17.000 | 17.000 |
| 2 | Sod. Chloride | — | — | 0.351 | — | — |
| 3 | Pot. Chloride | — | — | 0.049 | — | |
| 4 | Sod. bicarb | — | — | 0.179 | — | |
| 5 | Sucralose | 0.042 | 0.042 | 0.042 | 0.042 | 0.042 |
| 6 | Maltitol Syrup | 5.000 | — | — | — | |
| 7 | Pectin | — | — | — | — | 0.167 |
| 8 | Citric acid | 0.100 | 0.100 | 0.100 | — | |
| 9 | Orange shade color | 0.002 | 0.002 | 0.002 | — | |
| 10 | Red # 40 | 0.100 | — | — | — | 0.002 |
| 11 | Mint Flavor | — | | — | 0.100 | |
| 12 | Orange flavor | — | 0.100 | 0.100 | — | |
| 13 | Strawberry Flavor | — | — | — | — | 0.100 |
| 14 | Purified water | 0.100 | 2.000 | 1.66 | 2.000 | 2.00 |
| Total | | 22.344 | 19.244 | 15.614 | 19.142 | 19.311 |
| | Comments | PEG gets | Waxy taste | PEG gets | Waxy taste | Taste, Texture |
| | | separated | | separated | | and appearance |
| | | | | | | are not good |
| | | | | | | at all |

Procedure:
RB-115-091: Melt PEG 3350 in a SS container kept on a water bath at 80° C. till it forms a clear liquid. Dissolve Sucralose in it. Mix Maltitol syrup, orange color, orange flavor. Dissolve citric acid in water and add to the maltitol syrup solution containing the flavor and color. Add this mixture to the melted PEG solution and mix well to form homogeneous mixture. Pour into Silicon molds. Cool to set.
RB-115-092: Melt PEG 3350 in a SS container kept on a water bath at 80° C. till it forms a clear liquid. Dissolve Sucralose in it. Dissolve flavor, color and citric acid in water and add to the melted PEG 3350. Mix well. .
It forms a clear solution. Pour into the molds. Cool to set.
RB-115-093: Melt PEG 3350 in a SS container kept on a water bath at 80° C. till it forms a clear liquid. Dissolve Sucralose in it. Dissolve all the 3 salts, citric acid in water. It forms a hazy solution. Add this solution to the melted PEG solution. Pour in to molds. Cool to set.
RB-115-094: Melt PEG 3350 in a SS container kept on a water bath at 80° C. till it forms a clear liquid. Dissolve Sucralose in it. Add the mint flavor, add the water, mix well. Pour into the molds. Cool to set.
RB-115-095: Melt PEG 3350 in a SS container kept on a water bath at 80° C. till it forms a clear liquid. Dissolve Sucralose in it. Suspend the Pectin in it. Dissolve the color, flavor in water and add the bulk. Pour in to the molds. Cool to set.

### Non-Chocolate Bars (Grams Per Bar)

| Item | Ingredients | RB115-096 | RB115-097 | RB115-098 | RB115-099 | RB115-100 |
|---|---|---|---|---|---|---|
| 1 | PEG 3350 | 17.000 | 17.000 | 17.000 | 17.000 | 17.000 |
| 2 | Sucralose | — | | | | 0.042 |
| 3 | Glycerin | 1.666 | 1.666 | 2.083 | 3.666 | 3.000 |
| 4 | Sugar | — | 4.166 | 8.333 | — | — |
| 5 | Isomalt | — | — | — | 8.333 | — |
| 6 | Red # 40 | — | — | 0.002 | 0.002 | 0.001 |
| 7 | Vanilla Flavor | — | — | — | — | 0.050 |
| 8 | Strawberry Flavor | — | — | 0.100 | 0.100 | 0.100 |
| 9 | Purified water | — | — | — | — | — |
| | Total Wt | 18.166 | 22.832 | 27.578 | 29.102 | 20.193 |
| | Comments | Taste not | Taste not | Taste not | Bad taste | Waxy taste |
| | | good | good | good | | |

Procedure:
RB-115-096: Melt PEG 3350 in a container on a water bath at 80° C. till it forms a clear solution. Add glycerin and mix well Cool to 60° C. and pour into molds. Cool to set.
RB-115-097: Melt PEG 3350 in a container on a water bath at 80° C. till it forms a clear solution. Add glycerin and mix well. Suspend sugar and mix well. Pour into molds. Cool to set.
RB-115-098: Melt PEG 3350 in a container on a water bath at 80° C. till it forms a clear solution. Dissolve the color and flavor in glycerin and add to the bulk. Suspend the sugar and mix well. Pour into the molds and cool to set.
RB-115-099: Melt PEG 3350 in a container on a water bath at 80° C. till it forms a clear solution. Dissolve the color and flavor in glycerin and add to the bulk. Suspend Isomalt and mix well. Pour into molds and cool to set.
RB-115-100: Melt PEG 3350 in a container on a water bath at 80° C. till it forms a clear solution. Add sucralose and dissolve. Dissolve the color and flavor in glycerin and add to the bulk. Cool to 70°. Pour in to molds. Cool to set.

### Non-Chocolate Bars (Grams Per Bar)

| | | PEG 3350 alone | | PEG 3350 + Electrolytes |
|---|---|---|---|---|
| | | 17 gms | | 13.125 + 0.351 + 0.049 + 0.179 |
| Item | Ingredients | RB115-101 | RB115-102 | RB115-103 |
| 1 | PEG 3350 | 17.000 | 17.000 | 13.125 |
| 2 | Sucralose | 0.021 | 0.042 | 0.042 |
| 3 | Soy Lecithin | 0.333 | 1.000 | 1.000 |
| 4 | Sweetened | 15.000 | 15.000 | 13.000 |
| | Condensed milk | | | |
| 5 | Sod. Chloride | — | — | 0.351 |
| 6 | Pot. Chloride | — | — | 0.049 |
| 7 | Sod. bi. carb | — | — | 0.179 |
| 8 | Red# 40 | — | 0.002 | 0.002 |
| 9 | Vanilla Flavor | 0.100 | — | — |
| 10 | Strawberry Flavor | | 0.100 | 0.100 |
| | Total Wt | 32.450 | 33.144 | 27.848 |
| | Comments | Taste, Texture | Taste, Texture | Taste, Texture and Appearance |
| | | and Appearance | and Appearance | are good |
| | | are good | are good | |

Procedure:
RB115-101: Melt PEG in a container on a water bath at 80° C. till it forms a clear solution. Dissolve Sucralose in it. Add Lecithin and dissolve. Add Sweetened Condensed Milk and mix well maintaining the temperature at 80° C. for 20 minutes. Cool to 70° C., add the flavor and mix well. Pour into molds and cool to set.
RB115-102: Same as 101
RB115-103: Melt PEG in a container on a water bath at 80° C. till it forms a clear solution. Dissolve Sucralose in it. Add Lecithin and dissolve. Add Sweetened Condensed Milk and mix well maintaining the temperature at 80° C. for 20 minutes. Add and mix well the salt blend previously powdered and mixed. Cool to 70° C., add the flavor and mix well. Pour into molds and cool to set.

SUMMARY for Non-Chocolate bars for experiments 91-103: Maltitol syrup, Pectin and Glycerin were tried in the formulation and were unsuccessful. Subsequently, Sweetened Condensed Milk/Soy lecithin were tried and found to produce good soft tasty bars.

### Dark Chocolate Bar with Fiber

| Item | Ingredients | RB-115-106 Per bar (gms) |
|---|---|---|
| 1 | PEG 3350 | 17.000 |
| 2 | Soluble fiber - (Wheat | 6.000 |
| | Dextrin); (Nutriose ®) | |
| 3 | Sucralose | 0.042 |
| 4 | Purified water | 2.500 |
| 5 | Sod. Benzoate | 0.030 |
| 6 | Dark Chocolate (ADM) | 12.000 |
| 7 | Strawberry Flavor | 0.100 |
| | Total wt | 37.672 |
| | Comments | Good taste, Good texture |

Procedure:
Melt PEG in a container on a water bath at 80° C. till it forms a clear solution. Dissolve Sucralose in it. Dissolve Sod. Benzoate in a small quantity of water and add to the bulk. Add Chocolate chips and disperse well by mixing and continuous heating at 80° C. Add the Nutriose ® and disperse well by mixing. Add the flavor. Pour in to molds and cool to set

### Non-Chocolate bar with Fiber

| Item | Ingredients | RB-115-107 Per bar (gms) |
|---|---|---|
| 1 | PEG 3350 | 17.000 |
| 2 | Soluble fiber - Wheat | 6.000 |
| | Dextrin (Nutriose ®) | |
| 3 | Sucralose | 0.042 |
| 4 | Soy - Lecithin (WhC) | 1.000 |
| 5 | Sweetened Condensed Milk | 15.000 |
| 6 | Vanilla Flavor | 0.100 |
| 7 | Caramel Flavor | 0.050 |
| | Total Wt | 39.192 |
| | Comments | Good taste, Soft bar in texture |

Procedure:
Melt PEG in a container on a water bath at 80° C. till it forms a clear solution. Dissolve Sucralose in it followed by Lecithin. Mix well. Add Sweetened Condensed Milk and mix well. Stop heating. Add the flavors and mix well. Pour into molds and cool to set.

### Aspects and Embodiments of the Invention

Embodiment 1: An edible, solid composition, comprising: a laxative and a bulking agent wherein the composition is formed by heating the laxative and bulking agent together to form a fluid dispersion and cooling the fluid dispersion to form the edible solid composition, and wherein the edible solid composition has laxative properties.

Embodiment 2: The edible, solid composition according to embodiment 1 or embodiment 22 wherein the bulking agent is selected from the group consisting of corn syrup, sorbitol, sucrose, dextrose, invert sugar, ludiflash, chocolate, maltitol, high fructose syrup, rice syrup, barley malt syrup, glycerin, maltodextrins, starch hydrolysate, honey, molasses, soy lecithin, condensed milk, fat free sweetened condensed milk, evaporated milk, sweetened condensed milk and any combination thereof.

Embodiment 3: The edible, solid composition according to embodiments 1 or 2 or embodiment 22 further comprising a sweetening agent.

Embodiment 4: The edible, solid composition according to embodiment 3 or embodiment 22 wherein the sweetening agent is selected from the group consisting of sugar, sorbitol, saccharine, aspartame, acesulfame potassium, sucralose, isomalt, mannitol, stevia, cyclamate, dextrose, glucose, fructose, honey, high fructose syrup and any combination thereof.

Embodiment 5: The edible, solid composition according to any one of embodiments 1-4 or embodiment 22 wherein the laxative is selected from the group consisting of polyethylene glycol, castor oil, senna, bisacodyl, sodium pico sulfate, magnesium citrate, magnesium hydroxide (milk of magnesia), magnesium carbonate magnesium sulfate (Epsom salt), sodium sulfate, potassium sulfate, sodium chloride, potassium chloride, sodium bicarbonate, aloes, cascara sacrada, psyllium husk (Metamusil), guar gum, maltodextrins, inulin, docusate sodium, methyl cellulose, monobasic sodium phosphate, dibasic sodium phosphate, glycerin, sorbitol, mannitol, sodium potassium tartrate, mineral oil (liquid paraffin), lactulose, rhubarb, agar agar, figs (Syrup of Figs). L-glucose and any combination thereof.

Embodiment 6: The edible, solid composition according to embodiment 5 or embodiment 22 wherein the laxative is polyethylene glycol, magnesium citrate, inulin, castor oil or lactulose.

Embodiment 7: The edible, solid composition according to any one of embodiments 1-6 or embodiment 22 wherein the laxative comprises at least 40 wt % relative to the total weight of the composition.

Embodiment 8: The edible, solid composition according to any of embodiments 1, 2, 3, 4 or 7 or embodiment 22 wherein the laxative is polyethylene glycol and the polyethylene glycol is melted or its physical form is converted to a flowable viscosity by application of heat.

Embodiment 9: The edible, solid composition according to any one of embodiments 1-8 or embodiment 22 wherein the bulking agent is mixed with the melted or flowably viscous polyethylene glycol to form a flowable dispersion of ingredients and the flowable dispersion is placed into one or more molds and cooled to form the solid composition.

Embodiment 10: The edible, solid composition according to any one of embodiments 1-9 or embodiment 22 in the form of a bar, a lozenge, a chewable candy, or a chewable solid confection.

Embodiment 11: The edible, solid composition according to any one of embodiments 1-10 or embodiment 22 wherein the form is substantially homogeneous.

Embodiment 12: The edible, solid composition according to any one of embodiments 1-11 or embodiment 22 which remains solid at ambient temperature.

Embodiment 13: The edible, solid composition according to any one of embodiments 1-12 or embodiment 22 wherein the bulking agent is selected from the group consisting of soy lecithin, condensed milk, fat free sweetened condensed milk, evaporated milk, sweetened condensed milk and any combination thereof; and the laxative is selected from the group consisting of polyethylene glycol, magnesium citrate, lactulose and any combination thereof; and the composition is in the solid form of a bar or chewable candy.

Embodiment 14: The edible, solid composition according to embodiment 13 or embodiment 22 wherein the bulking agent is a combination of soy lecithin and a second bulking agent selected from the group consisting of condensed milk, fat free sweetened condensed milk, evaporated milk sweetened condensed milk and any combination thereof; the laxative is polyethylene glycol; and the composition is in the solid form of a bar or chewable candy.

Embodiment 15: The edible, solid composition according to any one of embodiments 1-14 or embodiment 22 further comprising an electrolyte.

Embodiment 16: The edible, solid composition according to any one of embodiments 1-15 or embodiment 22 further comprising a flavoring agent.

Embodiment 17: The edible, solid composition according to embodiment 15 or embodiment 22 wherein the electrolyte is selected from the group consisting of sodium chloride, potassium chloride, potassium bicarbonate, magnesium chloride and any combination thereof.

Embodiment 18: The edible, solid composition according to embodiment 16 or embodiment 22 wherein the flavoring agent is selected from the group consisting of bubble gum, cherry, mixed berry, fruit punch, pine apple, caramel, mixed fruit, chocolate, spearmint, peppermint, menthol licorice, orange, lemon, Watermelon, melagawine, cranberry, tutti frutti, mix of cardamom and orange, vanilla, ice cream soda, salt masking flavor and any combination thereof.

Embodiment 19: The edible, solid composition according to any of the foregoing embodiments or embodiment 22 wherein the laxative is polyethylene glycol is polyethylene glycol 3350.

Embodiment 20: The edible composition according to any of the foregoing embodiments or embodiment 22 wherein the bulking agents are less than 20% of the weight of the bar.

Embodiment 21: The edible, solid composition according to any of the foregoing embodiments or embodiment 22 wherein the bulking agents are less than 10% of the weight of the bar.

Embodiment 22: An edible solid composition comprising a substantially homogeneous, solid, amorphous dispersion of a laxative, a bulking agent and a sweeting agent, the dispersion having a smooth, non-crystalline, non-gritty mouth feel and a sweet taste.

Embodiment 23: The edible solid composition according to embodiment 22 wherein the laxative is polyethylene glycol magnesium citrate, inulin, castor oil or lactulose.

Embodiment 24: The edible solid composition according to embodiment 22 wherein the laxative is polyethylene glycol

Embodiment 25: The edible solid composition according to embodiment 22 containing chocolate.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An edible, solid composition, comprising: a laxative and a bulking agent wherein the composition is formed by heating the laxative and bulking agent together to form a fluid dispersion and cooling the fluid dispersion to form the edible solid composition, and wherein the edible solid composition has laxative properties.

2. The edible, solid composition according to claim 1 wherein the bulking agent is selected from the group consisting of corn syrup, sorbitol, sucrose, dextrose, invert sugar, ludiflash, chocolate, maltitol, high fructose syrup, rice syrup, barley malt syrup, glycerin, maltodextrins, starch hydrolysate, honey, molasses, soy lecithin, condensed milk, fat free sweetened condensed milk, evaporated milk, sweetened condensed milk and any combination thereof.

3. The edible, solid composition according to claim 1 further comprising a sweetening agent.

4. The edible, solid composition according to claim 3 wherein the sweetening agent is selected from the group consisting of sugar, sorbitol, saccharine, aspartame, acesulfame potassium, sucralose, isomalt, mannitol, stevia, cyclamate, dextrose, glucose, fructose, honey, high fructose syrup and any combination thereof.

5. The edible, solid composition according to claim 1 wherein the laxative is selected from the group consisting of polyethylene glycol, castor oil, senna, bisacodyl, sodium pico sulfate, magnesium citrate, magnesium hydroxide (milk of magnesia), magnesium carbonate magnesium sulfate (Epsom salt), sodium sulfate, potassium sulfate, sodium chloride, potassium chloride, sodium bicarbonate, aloes, cascara sacrada, psyllium husk (Metamusil), guar gum, maltodextrins, inulin, docusate sodium, methyl cellulose, monobasic sodium phosphate, dibasic sodium phosphate, glycerin, sorbitol, mannitol, sodium potassium tartrate, mineral oil (liquid paraffin), lactulose, rhubarb, agar agar, figs (Syrup of Figs), L-glucose and any combination thereof.

6. The edible, solid composition according to claim 5 wherein the laxative is polyethylene glycol, magnesium citrate, inulin, castor oil or lactulose.

7. The edible, solid composition according to claim 1 wherein the laxative comprises at least 40 wt % relative to the total weight of the composition.

8. The edible, solid composition according to claim 1 wherein the laxative is polyethylene glycol and the polyethylene glycol is melted or its physical form is converted to a flowable viscosity by application of heat.

9. The edible, solid composition according to claim 8 wherein the bulking agent is mixed with the melted or flowably viscous polyethylene glycol to form a flowable dispersion of ingredients and the flowable dispersion is placed into one or more molds and cooled to form the solid composition.

10. The edible, solid composition according to claim 1 in the form of a bar, a lozenge, a chewable candy, or a chewable solid confection.

11. The edible, solid composition according to claim 10 wherein the form is substantially homogeneous.

12. The edible, solid composition according to claim 11 which remains solid at ambient temperature.

13. The edible, solid composition according to claim 10 wherein the bulking agent is selected from the group consisting of soy lecithin, condensed milk, fat free sweetened condensed milk, evaporated milk, sweetened condensed milk and any combination thereof; and the laxative is selected from the group consisting of polyethylene glycol, magnesium citrate, lactulose and any combination thereof; and the composition is in the solid form of a bar or chewable candy.

14. The edible, solid composition according to claim 13 wherein the bulking agent is a combination of soy lecithin and a second bulking agent selected from the group consisting of condensed milk, fat free sweetened condensed milk, evaporated milk, sweetened condensed milk and any combination thereof; the laxative is polyethylene glycol; and the composition is in the solid form of a bar or chewable candy.

15. The edible, solid composition according to claim 1 further comprising an electrolyte.

16. The edible, solid composition according to claim 1 further comprising a flavoring agent.

17. The edible, solid composition according to claim 15 wherein the electrolyte is selected from the group consisting of sodium chloride, potassium chloride, potassium bicarbonate, magnesium chloride and any combination thereof.

18. The edible, solid composition according to claim 16 wherein the flavoring agent is selected from the group consisting of bubble gum, cherry, mixed berry, fruit punch, pine apple, caramel, mixed fruit, chocolate, spearmint, peppermint, menthol, licorice, orange, lemon, Watermelon, melagawine, cranberry, tutti frutti, mix of cardamom and orange, vanilla, ice cream soda, salt masking flavor and any combination thereof.

19. The edible, solid composition according to claim 1, wherein the laxative is polyethylene glycol is polyethylene glycol 3350.

20. The edible composition according to claim 1, wherein the bulking agents are less than 20% of the weight of the bar.

21. The edible, solid composition according to claim 1, wherein the bulking agents are less than 10% of the weight of the bar.

22. An edible solid composition comprising a substantially homogeneous, solid, amorphous dispersion of a laxative, a bulking agent and a sweeting agent, the dispersion having a smooth, non-crystalline, non-gritty mouth feel and a sweet taste.

23. The edible solid composition according to claim 22 wherein the laxative is polyethylene glycol, magnesium citrate, inulin, castor oil or lactulose.

24. The edible solid composition according to claim 22 wherein the laxative is polyethylene glycol.

25. The edible solid composition according to claim 22 containing chocolate.
